(19) United States
(12) Patent Application Publication
(10) Pub. No.: US 2003/0059487 A1
(43) Pub. Date: Mar. 27, 2003

(54) Composition and method for the treatment of hypercholesterolemia and hyperlipidemia in mammals

(76) Inventor: Sarfaraz K. Niazi, Deerfield, IL (US)

Correspondence Address: Welsh & Katz, Ltd., Irene M. Reininger, 22nd Floor, 120 South Riverside Plaza, Chicago, IL, 60606, US

(21) Appl. No.: 09/957,773

(22) Filed: Sep. 21, 2001

(51) Int. Cl. A61K 35/78

(52) U.S. Cl. 424/769; 424/777

(57) ABSTRACT

A pharmaceutical composition suitable for the treatment of a condition selected from the group consisting of atherosclerosis, post-angioplasty restenosis, coronary artery disease, angina, small artery disease and other diseases caused by or are the result of hypercholesterolemia, or combination thereof comprising of a specific botanical plant. Specifically, the present invention relates to compositions comprising the herb or botanical Zizyphus jujube or extract thereof, which is useful in treating cardiovascular disorders, particularly those associated with elevated LDL and overall serum cholesterol levels. A method of preparing the pharmaceutical compositions of the invention and a method for treating a patient therewith are also disclosed.

### FIELD OF THE INVENTION

This invention related to a composition and method for treating cardiovascular disease in humans. More particularly, the invention concerns the use of zizyphus jujuba fruit for reducing low-density level cholesterol, and plasma cholesterol in patients with, or at risk of developing cardiovascular disease associated with elevated cholesterol levels.

### BACKGROUND OF THE INVENTION

Cardiovascular disease is currently the leading cause of death in the United States despite a 33% decrease in the incidence of the disease over the past 20 years. Several causative factors are associated with cardiovascular disease, these include primarily infections, autoimmune response, hypercholesterolemia and hyperlipidemia. Hypercholesterolemia, in particular is an important risk factor linked with cardiovascular disease, and plays a significant role in the cause of atherosclerosis.

Approximately 90% of cardiovascular disease is diagnosed as atherosclerosis, which is characterized by the narrowing of the lumen and hardening of the arterial walls. Atherosclerosis is caused by high blood plasma concentrations of low-density lipoproteins, (LDL). LDL is a lipid, which carries most of the cholesterol and is the main source of damaging accumulation and blockage in the arteries. Specifically, LDL functions to transports cholesterol to peripheral tissues and regulates endogenous cholesterol levels in those tissues. LDL contains specific functional groups, which allow it to be recognized by most cells in the body and remain soluble in blood plasma. Consequently, LDL readily passes through the endothelium, contributing to the development of plaques, which over time accumulates and grows, causing arterial wall cholesterol. Therefore, patients with low levels of LDL rarely develop atherosclerosis. Conversely, the levels of HDL, or high-density lipoproteins significantly leads to the lowering of overall serum cholesterol in the body and greatly decreases the chances of cardiovascular disease. HDL, participates in the transport of cholesterol from peripheral tissues to the liver, which in turn leads to the elimination of cholesterol from the body. Other lipids such as chylomicrons, also function to transport cholesterol through the body. Chylomicrons specifically participates in the transporting of dietary triglycerides and cholesterol from the intestine to adipose tissue and liver. Triglycerides are a form of fat carried through the bloodstream. Most of the body's fat is in the form of triglycerides stored in fat tissue, and only a small portion of the triglycerides are found in the bloodstream. High blood triglycerides levels alone do not cause atherosclerosis, however lipoproteins that are rich in triglycerides also contain cholesterol, and may also lead to atherosclerosis.

Consequently, although the inventor does not wish to be bound by any particular theory of the invention, it is believed that by significantly lowering the lipoprotein, LDL, and cholesterol, one can lessen the risks and treat the effects of cardiovascular diseases. As such, over the years many different strategies have been employed to assist in the treatment of this disease. One such treatment is a combination of dietary supplements that have been shown to lower serum cholesterol, protect against LDL cholesterol oxidation, and reduce the risk of an abnormal arterial blood clot formation. For example, the supplementation of chromium to normal individuals has been reported to lead to improvements in glucose tolerance, serum lipid concentrations, including high-density lipoprotein cholesterol, insulin and insulin binding (Anderson, Clin. Psychol. Biochein. 4:31-41, 1986).

Additionally, there have been several drug classes that are commonly used to lower LDL levels. Drugs that inhibit the enzyme HMG-CoA reductase are referred to as “statins.” These drugs lower cholesterol by slowing down the production of cholesterol and by increasing the liver's ability to remove the LDL cholesterol already in the blood. The latest introduction to the powerful group of lipid-lowering drugs known as statins, or HMG reductase inhibitors, is atorvastatin (Lipitor). It is the only statin approved for the reduction of triglycerides as well as total and LDL cholesterol. It reduces LDL by 40 to 60%, triglycerides by 20 to 40%, and raises HDL cholesterol by 5 to 10%, changes which may be bigger than those produced by other statins. Other available statins which primarily reduce LDL cholesterols are Atorvastatin®, cerivastatin (Baychol®), fluvastatin (Lescol®), lovastatin (Mevacor®), pravastatin (Pravachol®) and simvastatin (Zocor®).

Another approach used to lower cholesterol includes pharmaceutical compositions which function to lower elevated triglyceride levels. These drugs include bile acid sequestrants (clofibrate), and nicotinic acid (niacin). Evidence suggests, however, that the atherogenic effects of low density lipoprotein (LDL) may be in part mediated through its oxidative modification. The problem with these triglyceride-lowering drugs, however, it that they have toxic side effects that cause many people to avoid them.

In addition to the use of conventional pharmaceutical therapies, traditional medicines (such as traditional Chinese medicines and traditional Japanese medicines) have been used for cardiovascular therapy for a number of years. These include the use of curcumin, also known as turmeric root, an ancient spice in the ginger family which is thought to reduce cholesterol in the body by blocking the formation of thromboxane A2, a promoter of platelet aggregation, thereby inhibiting abnormal blood clot formation. Curcumin also works to increase prostacyclin, a natural inhibitor of platelet aggregation. (Arzneim. Forsch., 1986, 36: 715-17). Consequently, curcumin is reported to demonstrate the ability to decrease total cholesterol and LDL cholesterol levels in serum and to increase the beneficial HDL cholesterol. Other herbal and botanical remedies found to treat cardiovascular disease by lowering cholesterol overall include Gugulipid, made from the resin of Commiphora Mukul tree in India, garlic, vitamin E, soy, high intake of soluble fiber, fish oil, and green tea. The problem however, is that a number of these naturally-derived, botanical and mineral products have some minor or significant side effects. As such, there is still a need and desire to identify natural sources that can be administered long term and provide a method for reducing plasma cholesterol in patients with, or at risk of developing such diseases associated with elevated cholesterol levels by safe and effective means.

Furthermore, other examples of herbal or botanical therapies that have been disclosed, include U.S. Pat. No. 5,589,182 to Tashiro, et al., a pharmaceutical composition suitable for the treatment of a condition selected from the group consisting of cardiovascular disease, cerebrovascular disease, Alzheimer's disease, depression or combinations thereof comprising various mixtures of the aqueous extracts of tissue of 21 specific Chinese plants and herbs. The U.S. Pat. No. 4,999,376 to Liu is for a composition for treating and preventing cardiovascular diseases comprising of Puerarin or derivate of Puerarin; Danshensu or Danshenketone; Tetramethyl pyrazine; Polysaccharides and Saponin of Dangshen. The U.S. Pat. No. 6,046,022 to Zhang, et al., is for a composition comprising of red rice fermentation products, that can be used as natural dietary supplements and/or medicaments for the treatment or prevention of hyperlipidemia and associated disorders and symptoms, such as cardiovascular diseases, cerebrovascular diseases, diabetes, hypertension, obesity, asthenic breathing, chronic headache, chest pain and tightness, limb swelling and distention, loss of appetite and excess expectoration.

### BRIEF SUMMARY OF THE INVENTION

It is an object of the present invention to provide a composition and method for treating cardiovascular disease by reducing plasma cholesterol in patients with, or at risk of developing such diseases associated with elevated cholesterol levels. The potential benefit is to reduce the side effects of typical therapies and provide a safe therapeutic that can be taken long term on a regular basis. The present invention is based on a known natural occurring botanical, which offers a surprisingly new therapy for treating cardiovascular disease.

Accordingly, there is provided a composition comprising: a botanical or herbal source, such as zizyphus that acts by reducing, elevated LDL and overall serum cholesterol levels in patients to safe and normal levels with, or at risk of developing cardiovascular disease.

These and other features and advantages of the present invention will be apparent from the following detailed description, in conjunction with the appended claims.

### DETAILED DESCRIPTION OF THE INVENTION

The present invention relates to the identification of a naturally occurring species from the botanical family Rhamnaceae family, and preferably from the genus Zizyphus, which when consumed on a regular basis, treats cardiovascular disease. Although such naturally occurring botanical sources have been traditionally utilized for a variety of treatments, it had not been, however, previously disclosed that the active herbal composition described herein, would produce a composition with the remarkable therapeutic effects for the prevention, treatment or amelioration of cardiovascular disease.

Specifically, the herbal or botanical plant employed in the present invention is in the genus Zizyphus, some species of which are as follows: Zizyphus vulgaris, Zizyphus. Lotos, Zizyphus. Jujuba, Zizyphus Baclei, Zizyphus Cenoplia, Zizyphus Barelei, Zizyphu. Napeca, Zizyphus. mauritiana, Zizyphus spina, Zizyphus mucronata Zizyphus nummularia, Zizyphus Spinosi, Zizyphus joazeiro, Zizyphus mistol, Zizyphus lotus, Zizyphus nummularia, Zizyphus nummularia, Zizyphus joazeiro, Zizyphus joazeiro, Zizyphus mucronata, Zizyphus sativa. In a preferred embodiment the invention comprises a composition which further includes at least one of the Zizyphus species listed above or extract thereof.

More particularly, the present invention utilizes the jujube berry, a fruit from the Zizyphus genus. This fruit is widely grown in South Asia and India in rather dry mild temperature conditions. In the preferred embodiment of the present invention the jujube fruit used is from the South Asian variety. The jujube is best described as a pectoral fruit in the same class as raisins, dates and figs. The smooth-leaved Chinese jujube (Ziziphus jujuba Mill) of the family Rhamnaceae, is of ancient culture in northern China. The jujube variety from South Asia is often merely called jujube, or Chinese date. Other English names are Indian Plum, Indian cherry and Malay jujube.

While the Indian jujube variety is cultivated to some extent throughout its natural range it is mostly grown commercially in the Indian subcontinent. There are over 90 cultivars of this fruit, differing in the habit of the tree, leaf shape, fruit form, size, color, flavor, keeping quality, and fruiting season. Among the important cultivars, eleven are described in the encyclopedia Wealth of India: Banarasi (or Banarsi) Pewandi, Dandan, Kaithli, Muria Mahrara, Narikelee, Nazuk, Sanauri 1, Sanauri 5, Thornless and Umran, Banarasi Karaka, Desi Alwar, Umran, Gola, Kaithli, Katha phal, Dandan, Gular Bashi, Kheera, Nazuk, Seo ber, Var. 1, 2, 3, 4, and 5.

The fruit is typically consumed fresh, dried, by Jujube paste, made from gum-arabic and sugar, mixed with orange-flower water, or through a decoction of the root. Particularly the Jujube paste may be dissolved in a decoction of jujubes and evaporated. In the Indian subcontinent, the ripe fruit are mostly consumed raw, but are sometimes stewed or candied. Residents of Southeast Asia eat the unripe fruits with salt. Ripe fruits crushed in water form a very popular cold drink. Acid types are used for pickling or for chutneys. In Africa, the dried and fermented pulp is pressed into cakes resembling gingerbread. Young leaves are cooked and eaten in Indonesia. In Venezuela, a jujube liqueur is made and sold as Crema de ponsigue. Seed kernels are eaten in times of famine.

It is not surprising therefore, that the jujube fruits have also been employed in a number of therapeutic ways over the years. These include the treatment of pulmonary ailments and fevers, or mixed with salt and chili peppers, for the treatment of indigestion and biliousness. The dried ripe fruit is a mild laxative. The seeds are sedative and are taken, sometimes with buttermilk to halt nausea, vomiting and abdominal pains in pregnancy. They assist in diarrhea and poulticed on wounds. Mixed with oils, they are rubbed on rheumatic areas. The leaves are applied as poultices and are helpful in liver troubles, asthma and fever and together with catechu, and administered when an astringent is needed as on wounds. The bitter astringent bark decoction is taken to halt diarrhea and dysentery and relieve gingivitis. The bark past is applied to sores. The root is purgative. A root decoction is given as a febrifuge, taenicide and emmenagogue. And the powdered root is dusted on wounds. Juice of the root bark is said to alleviate gout and rheumatism. Strong doses of the bark or root may be toxic. An infusion of the flowers serves as an eye lotion.

The therapeutic composition disclosed in the present invention however, exhibits the highly advantageous combination of being highly effective in the treatment of cardiovascular disease, relatively inexpensive and of low toxicity. Specifically, the herbal composition of the invention is remarkably effective against cardiovascular disease including but not limited to athererosclerosis, post-angioplasty restenosis, coronary artery disease, angina, and small artery disease or other diseases caused by or as a result of hypercholesterolemia. In particular the present invention relates to a composition of an herbal or botanical source that when consumed regularly significantly lower the low-density lipids and cholesterol in the body if these levels are elevated above what is safe and normal.

The dosage however administered to a particular patient will depend upon a variety of conditions, i.e., the disease(s) under treatment and the severity thereof, the age and condition of the patient, etc. Generally, however, a dosage ranging from 1 to 10 fruits per day, preferably from about 3-6 fruits and more preferably 5 fruits a day. The composition is preferably taken 1-3 times per day and more preferably 2-3 times per day. The herbal composition when taken for about 12-18 weeks reduces the blood cholesterol from 8-25%, preferably from about 10-23% and more preferably about 20%. The total lipid reduction after treatment with the herbal composition of the present invention ranges from about 15-25%, and sometimes 18-23%.

The herbal composition can be prepared by a number of formulations, varying from consuming the fruit alone, soaked jujube, candied jujube, in capsule form, in a decoction drink or by organic extract. With respect to the soaked jujube fruit preparation the fresh ripe fruit is immersed in fresh water either at room temperature or a temperature near boiling with a ratio of water to fruit from about 2 parts water weight by weight to about 10 parts water weight by weight to about 1 part fruit. After immersing the fruit in water, the fruit is soaked for a period of time allowing for the fruit to be absorbed by the water, preferably overnight.

An organic extract of the fruit for the composition may also be used. The jujube fruit is ground, minced or crushed in an appropriate manner and extracted using as the solvent water, hydrophilic organic solvents or a mixture thereof. Organic solvents used for extraction are most appropriately especially, lower alcohols such as methanol or ethanol. The method of extraction is not particularly restricted, and the active ingredients can be extracted by immersing in the solvent maintained at room temperature to a temperature near the boiling point.

In the present invention the herbal or botanical composition may also be consumed/administered by a variety of means and may utilize different parts of the whole plant. In particular the fruit (whole or in part), stem, bark, seed, kernel, pit, pollen, leaf or root can be used in the preparation. The composition can employ either fresh or dried forms or in the form of an aqueous, alcoholic or other organic extract. The herbal composition can combine the different forms of the herb. The herbal or botanical composition may be administered orally, topically, or by rectum with an intended delivery to either the alimentary canal or absorption of active components at the site of administration. The oral composition can be administered in a pharmaceutical dosage form suitable for enteral administering to humans such as tablets, capsules, syrups, liquid, suspension, liposomal preparation, or powder.

Furthermore, the herbal composition can be used for the treatment of cardiovascular disease alone or in combination with a pharmaceutical cardiovascular agents including but not limited to lipid lowering agent, platelet aggregation inhibitors, antithrombotic agents, calcium channel blockers, angiotensin converting enzyme (ACE) inhibitors and β-blockers.

The mechanism by which the herbal or botanical composition of the present invention works is not presently known. It is theorized, however, that the composition of this invention work by its ability to decrease total cholesterol and LDL cholesterol levels in serum and to increase the beneficial HDL cholesterol.

While the present invention is susceptible of embodiment in various forms, there is shown in the examples and will hereinafter be described a presently preferred embodiment with the understanding that the present disclosure is to be considered an exemplification of the invention and is not intended to limit the invention to the specific embodiment illustrated. It should be further understood that the title of this section of this specification, namely, “Detailed Description Of The Invention”, relates to a requirement of the United States Patent Office, and does not imply, nor should be inferred to limit the subject matter disclosed and claimed herein.

### WORKING EXAMPLES

### Example 1

Fresh fruits, almost ripe, pits removed, are washed thoroughly with water to remove any debris, the stem stubs are removed, and fruits are consumed with pits removed; 5-10 fruits are consumed two to three times per day. The physico-chemical composition of the product per 100 G of the product is approximately:

| | | |
|---|---|---|
| Moisture | 81.6-83.0 | g |
| Protein | 0.8 | g |
| Fat | 0.07 | g |
| Fiber | 0.60 | g |
| Carbohydrates | 17.0 | g |
| Total Sugars | 5.4-10.5 | g |
| Reducing Sugars | 1.4-6.2 | g |
| Non-Reducing Sugars | 3.2-8.0 | g |
| Ash | 0.3-0.59 | g |
| Calcium | 25.6 | mg |
| Phosphorus | 26.8 | mg |
| Iron | 0.76-1.8 | mg |
| Carotene | 0.021 | mg |
| Thiamine | 0.02-0.024 | mg |
| Riboflavin | 0.02-0.038 | mg |
| Niacin | 0.7-0.873 | mg |
| Citric Acid | 0.2-1.1 | mg |
| Ascorbic Acid | 65.8-76.0 | mg |
| Fluoride | 0.1-0.2 | ppm |
| Pectin (dry basis) | 2.2-3.4% | |

| | |
|---|---|
| Malic acid | Traces |
| Oxalic acid | Traces |
| Quercetin | Traces |

Twelve adults whose cholesterol levels were between 230 and 300 consumed the above-mentioned quantities for 12 weeks; on an average, the reduction in blood cholesterol was about 20%. Eight subjects had total lipids reduced by about 23%.

### Example 2

Soaked Jujube: Fresh fruits, almost ripe, are washed thoroughly with water to remove any debris, the stem stubs are removed and fruits are immersed in fresh water at room temperature with a 1 one part of fruit to 10 parts of water weight by weight. The fruits are set aside overnight and consumed the following day, with pits removed; 5-10 fruits are consumed 2-3 times per day.

### Example 3

Candied Jujube: Fresh fruits, pits removed, slightly under-ripe, are washed thoroughly with water to remove any debris, the stem stubs are removed and the fruits are pricked with fork or a similar object lightly. The fruits are then immersed in a 2% salt solution for 24 hours; the next day, they are transferred to 3% salt solution, raising the concentration of salt by 1% each day until the final concentration of 8% salt is reached; after leaving the fruits in the 8% solution for 24 hours, the liquid is drained and the fruits immersed in another 8% salt solution containing 0.2% potassium metabisulfite. This solution containing the fruits is then stored for 1 to 3 months at ambient room temperature. After a minimum of one month, the fruits are removed, rinsed with water, and cooked in sugar syrup containing citric acid, about 1%. Candied fruits are consumed, 5-10 fruits, 2-3 times per day.

### Example 4

Fresh fruits, almost ripe, pits removed, are washed thoroughly with water to remove any debris, the stem stubs are removed, and fruits are dried under vacuum after prickling their surface at 60 degree centigrade until the total amount of moisture left in the fruits is less than 5%. The fruits are then powdered in a suitable mill and the powder filled in hard gelatin capsules; 0.2% metabisulfite is used before filling as stabilizer. Suitable lubricants such as microcrystalline cellulose is added depending on the equipment requirement for filling the product into capsules, preferably size zero. In most instances, one capsules is equivalent to one fruit content and thus about 5 capsules are taken 1-3 times per day. The composition of the powder per 100 G is as follows:

| | | |
|---|---|---|
| Calories | 104 | |
| Moisture | 5 | g |
| Protein | 4.12 | g |
| Fat | 0.53 | g |
| Carbohydrates | 7.21 | g |
| Sugar | 61.0 | g |
| Fiber | 3.44 | g |

### Example 5

Fresh fruits, almost ripe, pits removed, are washed thoroughly with water to remove any debris, the stem stubs are removed, and fruits are boiled in fresh water for about 10 to 15 minutes after crushing them. After cooling down, the decoction is consumed either directly or after straining the pulp. The decoction drink may be prepared fresh once a day and kept refrigerated until used. The quantity of decoction consumed is equivalent to about 5 fruits each time for 1-3 times per day.

### Example 6

Fresh fruits, almost ripe, pits removed, are washed thoroughly with water to remove any debris, the stem stubs are removed, and fruits are crushed and soaked in Ethanol USP for about three weeks covered at ambient temperature. After three weeks period, Ethanol USP solution is strained first through muslin cloth to remove pulp, which is then squeezed or compressed to extract more alcohol out of it, the portion being mixed with the strained liquid. Many techniques commonly available in the pharmaceutical industry can be used to remove the solids from liquid as intended here. The ethanolic solution is then evaporated at elevated temperature under vacuum until a dry residue is obtained. The powdered residue is mixed with a suitable stabilizer such as 0.2% metabisulfite and other excipients necessary for the filling of powder into capsules are added. The size of capsule is chosen such that each capsules represents content of about five fruits. One capsule is administered 1-3 times per day.

In the present disclosure, the words “a” or “an” are to be taken to include both the singular and the plural. Conversely, any reference to plural items shall, where appropriate, include the singular.

From the foregoing it will be observed that numerous modifications and variations can be effectuated without departing from the true spirit and scope of the novel concepts of the present invention. It is to be understood that no limitation with respect to the specific embodiments illustrated is intended or should be inferred. The disclosure is intended to cover by the appended claims all such modifications as fall within the scope of the claims.

1. A method for treating a patient comprising, administering a therapeutic amount of the herbal composition of the Zizyphus jujube effective to prevent or ameliorate the effects of a condition selected from the group consisting of atherosclerosis, post-angioplasty restenosis, coronary artery disease, angina, and small artery disease or other disease caused by or as a result of hypercholesterolemia and hyperlipidemia.

2. A method according to claim 1, wherein said Zizyphus jujube or extract thereof is effective in reducing low-density cholesterol and plasma cholesterol in patients with, or at risk of developing cardiovascular disease associated with elevated cholesterol levels.

3. A method according to claim 1, wherein said Zizyphus jujube is the jujube fruit.

4. A method of claim 1, wherein said herbal composition is administered orally to a subject.

5. An herbal composition mixture comprising, Zizyphus jujube, wherein the weight to water of said Zizyphus jujube tissues is in the range of from about 2:1 to about 10:1.

6. An herbal composition mixture comprising, Zizyphus jujube, wherein said Zizyphus jujube is administered fresh, dried, from extract, soaked, candied, in capsule or tablet form, orally, rectally, topically or in a decoction drink.

7. An herbal composition according to claim 6, wherein said extract is in the form of aqueous, alcoholic or organic extract.

8. A pharmaceutical composition for the treatment of cardiovascular disease comprising:
a therapeutically effective amount of an herbal composition comprising of Zizyphus jujube,
said herbal composition combined with a selected pharmaceutical cardiovascular agent.

9. A pharmaceutical composition according to claim 8, wherein said herbal composition is the jujube fruit.

10. A pharmaceutical composition according to claim 8, wherein said pharmaceutical cardiovascular agent is selected from the group consisting of lipid lowering agent, platelet aggregation inhibitors, antithrombotic agents, calcium channel blockers, angiotensin converting enzyme inhibitors and β-blockers.

11. A pharmaceutical composition according to claim 8, wherein said combination comprising herbal composition with a pharmaceutical cardiovascular agent is orally administered to a subject.

12. A pharmaceutical composition according to claim 8, wherein said combination comprising herbal composition with a pharmaceutical cardiovascular agent is rectally administered to a subject.

13. A pharmaceutical composition according to claim 8, wherein said combination comprising herbal composition with a pharmaceutical cardiovascular agent is topically administered to a subject.

14. A pharmaceutical composition according to claim 8, wherein said cardiovascular disease is selected from the group consisting of atherosclerosis, post-angioplasty restenosis, coronary artery disease, angina, and small artery disease or other disease caused by or as a result of hypercholesterolemia and hyperlipidemia.
